(19) United States
(12) Patent Application Publication
(10) Pub. No.: US 2020/0325547 A1
(43) Pub. Date: Oct. 15, 2020

(54) METHOD FOR IDENTIFYING STORABILITY OF APPLE FRUIT AND SPECIFIC PRIMER PAIR THEREOF

(71) Applicant: Shandong Agricultural University, Shandong (CN)

(72) Inventors: Zongying ZHANG, Shandong (CN); Yicheng WANG, Shandong (CN); Cuicui WANG, Shandong (CN); Zuolin MAO, Shandong (CN); Nan WANG, Shandong (CN); Mengyu SU, Shandong (CN); Jing ZHANG, Shandong (CN); Shenghui JIANG, Shandong (CN); Xuesen CHEN, Shandong (CN)

(73) Assignee: Shandong Agricultural University, Shandong (CN)

(21) Appl. No.: 16/688,095

(22) Filed: Nov. 19, 2019

(30) Foreign Application Priority Data
Apr. 12, 2019 (CN) 201910292763.0

(51) Int. Cl. C12Q 1/6895; C12Q 1/6827

(52) U.S. Cl. CPC C12Q 1/6895; C12Q 2600/13; C12Q 1/6827

(57) ABSTRACT

The present invention discloses a method for identifying fruit storability of apple fruit and a specific primer pair. The specific primer pair includes primers F and R; the primer F is a single-stranded DNA molecule depicted in SEQ ID No.1, and the primer R is a single-stranded DNA molecule depicted in SEQ ID No.2. It has been demonstrated that the specific primer pair can genotype ACS1 gene of an apple plant to further determine the fruit storability, decreasing costs of land, manpower and material resources. The present invention is of great importance in improving breeding efficiency and reducing breeding cost and has great application value in apple breeding.

### CROSS-REFERENCE TO RELATED APPLICATION

This application claims priority to Chinese Patent Application No. 201910292763.0 filed on Apr. 12, 2019, the entire contents of which are herein incorporated by reference.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created Dec. 27, 2019, is named “Untitled_ST25” and is 30 kB in size.

### TECHNICAL FIELD

The present invention belongs to the field of plant molecular breeding, and in particular to a method for identifying the storability of apple fruit and a specific primer pair thereof.

### BACKGROUND

Apple industry plays a critical role in enriching farmers and promoting rural development. The variety structure in the apple industry is not quite reasonable in China at present, because the production of ‘Red Fuji’ apple accounts for at least 70% of the total apple yield. The issue of single cultivar is becoming more and more prominent, and the overcapacity of a single apple cultivar aggravates the continuing decline of apple fruit price. Therefore, it is urgent to have diversified excellent varieties for updating in production.

An excellent fresh apple cultivar should have traits of good appearance, crispness, juiciness, rich aroma, and long storage period.

Apple is a complex polyploidy tree species, and traits of its hybrid progenies are widely segregated, with difficult breeding, high cost, and long cycle; in particular, when integrating multiple quality traits, trait segregation in progenies will become more diverse and difficult. So far, molecular biological technique-assisted breeding, i.e., targeted selection of crossing parents through crossing parent analysis, is of great significance in improving breeding efficiency and reducing breeding cost.

### SUMMARY

The objective of the present invention is to identify storability of apple fruit.

First of all, the present invention provides a specific primer pair, including primers F and R;

the primer F can be the following a1) or a2):

a1) a single-stranded DNA molecule depicted in SEQ ID No.1;

a2) a DNA molecule acquired by substitution of SEQ ID No.1 with one or more nucleotides and/or deletion and/or addition and with identical function to SEQ ID No.1; and

the primer F can be the following a3) or a4):

a3) a single-stranded DNA molecule depicted in SEQ ID No.2; and

a4) a DNA molecule acquired by substitution of SEQ ID No.2 with one or more nucleotides and/or deletion and/or addition and with identical function to SEQ ID No.2.

An application of the specific primer pair in screening the storability of apple fruit also pertains to the scope of the protection of the present invention.

The present invention further provides a method for identifying fruit storability of apple fruit, including the following steps:

with genomic DNA of test apple as template, conducting PCR amplification by means of the specific primer pair, to obtain a PCR amplified product(s): if there is one type of 655 bp PCR amplified product, the genotype of the test apple plant is homozygous for ACS1-2/-2; if there are two types of PCR amplified products of 517 bp and 655 bp, respectively, the genotype of the test apple plant is heterozygous for ACS1-1/-2; if there is one type of 517 bp PCR amplified product, the genotype of the test apple plant is homozygous for ACS1-1/-1;

apple plant homozygous for ACS1-2/-2 has greater fruit storability than that heterozygous for ACS1-1/-2, followed by that homozygous for ACS1-1/-1.

The test apple plant can be any of the following apple cultivars: Malus domestica ‘Delicious’, Malus domestica ‘Ralls Janet’, Malus domestica ‘Royal Gala’, or Malus domestica ‘Mato’.

The test apple plant can be a hybrid progeny of any of the following apple cultivars: Malus domestica ‘Delicious’, Malus domestica ‘Ralls Janet’, Malus domestica ‘Royal Gala’, or Malus domestica ‘Mato’.

The test apple plant can be a hybrid progeny of Malus domestica ‘Delicious’×Malus domestica ‘Ralls Janet’ plants

The test apple plant can be a hybrid progeny of Malus domestica ‘Royal Gala’×Malus domestica ‘Mato’.

An application of the specific primer pair in identifying the storability of apple fruit also pertains to the scope of the protection of the present invention.

An application of the specific primer pair in apple breeding is disclosed. The objective of the breeding is to obtain an apple cultivar with excellent fruit storability.

The present invention further provides a DNA molecule, which can present a target sequence of the specific primer pair in apple genome.

An application of the DNA molecule in screening apple fruit with fruit storability also pertains to the scope of the protection of the present invention.

The present invention further provides a method for identifying storability of apple fruit, including the following steps: detecting whether there are DNA fragments depicted in SEQ ID No.3 and SEQ ID No.4 in the genomic DNA of a test apple and then determining as follows: if there is a DNA fragment depicted in SEQ ID No.3, but not in SEQ ID No.4, in the genomic DNA of the test apple, then the genotype of the test apple plant is homozygous for ACS1-2/-2; if there are DNA fragments depicted in both SEQ ID No.3 and SEQ ID No.4 in the genomic DNA of the test apple, then the genotype of the test apple plant is heterozygous for ACS1-1/-2; if there is a DNA fragment depicted in SEQ ID No.4, but not in SEQ ID No.3, in the genomic DNA of the test apple, then the genotype of the test apple plant is homozygous for ACS1-1/-1;

the fruit storability of apples with homozygous for ACS1-2/-2 genotype is better than those with ACS1-1/-2 and ACS1-1/-1 genotype.

the apple plant homozygous for ACS1-2/-2 has greater fruit storability than that heterozygous for ACS1-1/-2, followed by that homozygous for ACS1-1/-1.

The test apple plant can be any of the following apple cultivars: Malus domestica ‘Delicious’, Malus domestica ‘Ralls Janet’, Malus domestica ‘Royal Gala’, or Malus domestica ‘Mato’.

The test apple plant can be a hybrid progeny of any of the following apple cultivars: Malus domestica ‘Delicious’, Malus domestica ‘Ralls Janet’, Malus domestica ‘Royal Gala’, or Malus domestica ‘Mato’.

The test apple plant can be a hybrid progeny of Malus domestica ‘Delicious’×Malus domestica ‘Ralls Janet’ plants.

The test apple plant can be a hybrid progeny of Malus domestica ‘Royal Gala’×Malus domestica ‘Mato’.

An application of the DNA molecule in identifying the storability of apple fruit also pertains to the scope of the protection of the present invention.

An application of the DNA molecule in apple breeding also pertains to the scope of the protection of the present invention. The breeding objective is to obtain an apple cultivar with excellent fruit storability.

In any of the foregoing applications, hybridize two cultivars of apple plants with specific genotypes, and screen out a plant where the genotype is homozygous for ACS1-2/-2 from hybrid progenies, i.e., the apple plant with excellent fruit storability is obtained.

In any of the foregoing applications, hybridize two cultivars of apple plants with specific genotypes, at least one of which has an expected trait, and screen out a plant where the genotype is homozygous for ACS1-2/-2 with the expected trait from hybrid progenies, i.e., the apple plant with excellent fruit storability and the expected trait is obtained.

In any of the foregoing applications, hybridize an apple plant with a specific genotype and trait A with that with a specific genotype and trait B to obtain an apple plant with both traits A and B. The trait A can be excellent fruit storability. The trait B can be firm, crisp, and juicy fruit, delicious taste with slight sweetness and sour, rich aroma, moderately sweet and sour taste, fine and crisp flesh, and the forth.

Any of the foregoing “>” can be statistically “>”.

Hereinbefore, the genomic DNA of the test apple is acquired by extracting leaves of the test apple plant (e.g., seedling leaves) or the flesh of the fruit of the test apple. In apple breeding, because of a long juvenile period (approximately 3 to 5 years) of the apple seedling, using leaves of the test apple plant (e.g., seedling leaves) as materials usually saves more costs of land, manpower and material resources.

It has been demonstrated that the method of the present invention can be used to identify the storability of apple fruit and further improve breeding efficiency, which is of great importance in reducing breeding cost. The present invention has great application value in apple breeding.

### BRIEF DESCRIPTION OF THE DRAWINGS

FIG. 1 shows genotyping results of plants of Malus domestica ‘Delicious’, Malus domestica ‘Ralls Janet’, a hybrid progeny of Malus domestica ‘Delicious’×Malus domestica ‘Ralls Janet’, Malus domestica ‘Royal Gala’, Malus domestica ‘Mato’, and a hybrid progeny of Malus domestica ‘Royal Gala’×Malus domestica ‘Mato’.

FIG. 2 shows storability test results of a hybrid progeny of Malus domestica ‘Delicious’×Malus domestica ‘Ralls Janet’.

### DETAILED DESCRIPTION

The following embodiment contributes to a better understanding of the present invention, but the present invention is not limited thereto.

All experimental methods in the following embodiment, unless otherwise specified, are conventional.

All experimental materials used in the following embodiment, unless otherwise specified, are purchased from conventional biochemical reagent stores.

Record and literature of Malus domestica ‘Delicious’: Yu ZF. Effects of sales conditions on the rate of quality deterioration of ‘McIntosh’ and ‘Delicious’ apples stored in controlled atmosphere. Journal of Nanjing Agricultural University, 1990,13(2):127-131. ‘Delicious’ apple has the following traits: thick peel, rich aroma, and unstorability.

Record and literature of Malus domestica ‘Ralls Janet’: Liu X J, Feng B C, Feng S Q, et al. Studies on anthocyanin biosynthesis and activities of related enzymes of ‘Ralls’ and its bud mutation. Acta Horticulturae Sinica, 2009,36(9):1249-1254. ‘Ralls Janet’ apple has the following traits: storability, delicious taste with slight sweetness and sour, firm, crisp, and juicy fruit, poor coloration, and strong stress resistance.

Record and literature of Malus domestica ‘Royal Gala’: Li H F, Luan C H, Liu J F, et al. Preliminary report of evaluation of four mid-early mature apple cultivars[J]. Delicious Fruits, 2009,41(1):9-10. ‘Royal Gala’ apple has the following traits: mid-early maturity, good coloration, and storability.

Record and literature of Malus domestica ‘Mato’: Li Z, Fan Y L. A high-yield cultivation technique of ‘Mato’ apple. Hebei Forestry, 2010(3):33-33. ‘Mato’ apple has the following traits: early maturity, good coloration, fine and crisp flesh, moderately sweet and sour taste, rich flavor, and unstorability.

### Embodiment

1. Establishment of a method for identifying storability of apple fruit

Inventors of the present invention established a method for identifying storability of apple fruit through a great deal of experiments. Detailed steps are as follow:

with genomic DNA of test apple (flesh of fruits or leaves of plants) as template, conducting PCR amplification by means of the specific primer pair composed of primers F (5′-AGAGAGATGCCATTTTTGTTCGTAC-3′, SEQ ID No.1) and R (5′-CCTACAAACTTGCGTGGGGATTATAAGTGT-3′, SEQ ID No.2), to obtain a PCR amplified product(s): if there is one type of 655 bp PCR amplified product, the genotype of the test apple plant is homozygous for ACS1-2/-2; if there are two types of PCR amplified products of 517 bp and 655 bp, respectively, the genotype of the test apple plant is heterozygous for ACS1-1/-2; if there is one type of 517 bp PCR amplified product, the genotype of the test apple plant is homozygous for ACS1-1/-1;

the apple plant homozygous for ACS1-2/-2 has greater fruit storability than that heterozygous for ACS1-1/-2, followed by that homozygous for ACS1-1/-1.

PCR amplified products were sequenced. Sequencing results showed that the nucleotide sequence of the 655 bp PCR amplified product was depicted in SEQ ID No.3 and that of the 517 bp PCR amplified product was depicted in SEQ ID No.4.

2. Verification Test 1

1) Obtaining Seedling

i) Emasculated Malus domestica ‘Ralls Janet’ was pollinated with pollens of Malus domestica ‘Delicious’ to obtain hybrid seeds.

ii) The hybrid seeds obtained in Step i) were sown and grew to obtain seedlings.

2) Screening Seedlings

i) Test apple plants were genotyped in accordance with the method established in Step 1. The test apple plants were Malus domestica ‘Delicious’ plants, Malus domestica ‘Ralls Janet’ plants, or seedlings obtained in Step 1).

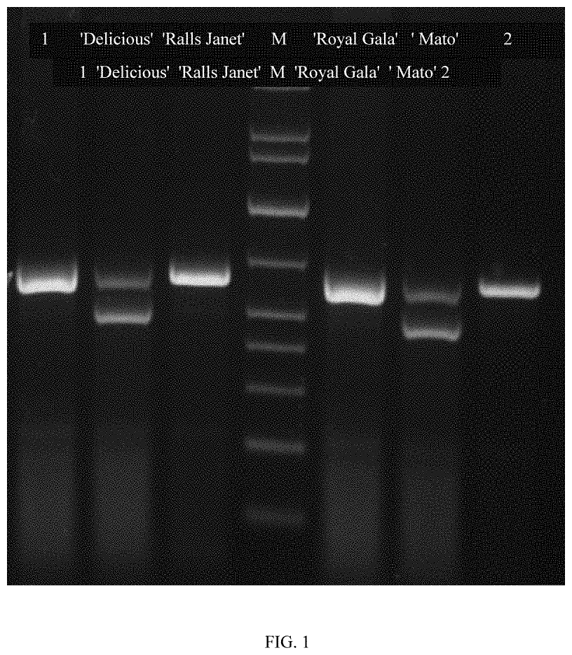

Some test results are shown in FIG. 1 (1 presents a seedling of Malus domestica ‘Delicious’×Malus domestica ‘Ralls Janet’). Results are as follows:

the genotype of the Malus domestica ‘Delicious’ plant is homozygous for ACS1-1/-2;

the genotype of the Malus domestica ‘Ralls Janet’ plant is heterozygous for ACS1-2/-2;

for seedlings obtained in Step 1), genotypes of one part of seedlings are heterozygous for

ACS1-1/-2, while those of the other part are homozygous for ACS1-2/-2.

ii) Apple plants with traits of delicious taste with slight sweetness and sour, firmness, crispiness, juiciness, and rich aroma, and homozygous for ACS1-2/-2 (named hybrid progeny of Malus domestica ‘Delicious’×Malus domestica ‘Ralls Janet’) were selected for storability test.

3) Storability Test

Flesh firmness was determined for test apples (Malus domestica ‘Delicious’, Malus domestica ‘Ralls Janet’, or the hybrid progeny of Malus domestica ‘Delicious’×Malus domestica ‘Ralls Janet’) by means of the method recorded in literature (Liu C C, Wei J L, Xu Y T, et al. Preliminary study on firmness and related physiological indices of three early ripening apple cultivar during late development of the fruit. Acta Horticulturae Sinica, 2011,38(1):133-138.). The fruit of each test apple was determined in duplicate and averaged, and each determination included the following steps of: harvesting fruits of the test apples, then storing at 4° C. for 10, 60, 90, 120, 150, 180, or 210 days, peeling at the fruit equator, and using a GY-3 Fruit Hardness Tester determine the flesh firmness.

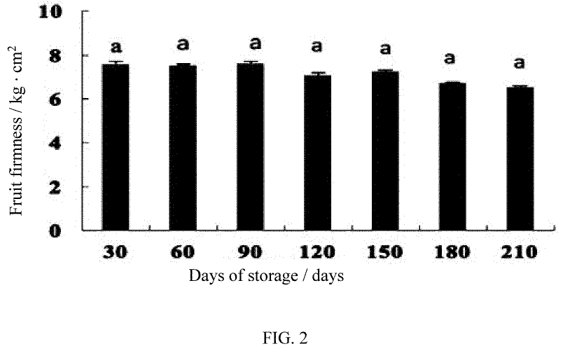

Test results of the hybrid progeny of Malus domestica ‘Delicious’×Malus domestica ‘Ralls Janet’ are illustrated in FIG. 2. Results showed that: there was no significant difference in flesh firmness of the hybrid progeny of Malus domestica ‘Delicious’×Malus domestica ‘Ralls Janet’ as days of storage increased; there was no significant difference in flesh firmness of Malus domestica ‘Ralls Janet’ (storable) as days of storage increased; the flesh firmness of Malus domestica ‘Delicious’ decreased significantly (unstorable) as days of storage increased.

3. Verification Test 2

1) Obtaining Seedling

i) Emasculated Malus domestica ‘Mato’ was pollinated with pollens of Malus domestica ‘Royal Gala’ to obtain hybrid seeds.

ii) The hybrid seeds obtained in Step i) were sown and grew to obtain seedlings.

2) Screening Seedlings

i) Test apple plants were genotyped in accordance with the method established in Step 1. The test apple plants were Malus domestica ‘Royal Gala’ plants, Malus domestica ‘Mato’ plants, or seedlings obtained in Step 1).

Some test results are shown in FIG. 1 (2 presents a seedling of Malus domestica ‘Royal Gala’×Malus domestica ‘Mato’). Results are as follows:

the genotype of the Malus domestica ‘Mato’ plant is heterozygous for ACS1-1/-2;

the genotype of the Malus domestica ‘Ralls Janet’ plant is homozygous for ACS1-2/-2;

for seedlings obtained in Step 1), genotypes of one part of seedlings are heterozygous for ACS1-1/-2, while those of the other part are homozygous for ACS1-2/-2.

ii) Apple plants with traits of good coloration, moderately sweet and sour taste, and fine and crisp flesh, and homozygous for ACS1-2/-2 (named hybrid progeny of Malus domestica ‘Royal Gala’×Malus domestica ‘Mato’) were selected for storability test.

3) Storability Test

Flesh firmness was determined for test apples (Malus domestica ‘Royal Gala’, Malus domestica ‘Mato’, or the hybrid progeny of Malus domestica ‘Royal Gala’×Malus domestica ‘Mato’) by means of the method recorded in literature (Liu C C, Wei J L, Xu Y T, et al. Preliminary study on firmness and related physiological indices of three early ripening apple cultivar during late development of the fruit. Acta Horticulturae Sinica, 2011,38(1):133-138.). The fruit of each test apple was determined in duplicate and averaged, and each determination included the following steps of: harvesting fruits of the test apples, then storing at 4° C. for 30, 60, 90, 120, 150, 180, or 210 days, peeling at the fruit equator, and using a Fruit Hardness Tester (GY-3) determine the flesh firmness.

Results showed that: there was no significant difference in flesh firmness of the hybrid progeny of Malus domestica ‘Delicious’×Malus domestica ‘Ralls Janet’ as days of storage increased; the flesh firmness of Malus domestica ‘Royal Gala’ decreased slowly (storable) as days of storage increased; the flesh firmness of Malus domestica ‘Mato’ decreased significantly (unstorable) as days of storage increased.

The foregoing results indicated that the method provided in Step 1 could identify the storability of apple fruit. The method provided in Step 1 could be applied in apple breeding.

What is claimed is:

1. A specific primer pair, comprising primers F and R,
wherein the primer F is:
a1) a single-stranded DNA molecule depicted in SEQ ID No.1; or
a2) a DNA molecule acquired by substitution of SEQ ID No.1 with one or more nucleotides and/or deletion and/or addition and with identical function to SEQ ID No.1; and
the primer F is:
a3) a single-stranded DNA molecule depicted in SEQ ID No.2; or
a4) a DNA molecule acquired by substitution of SEQ ID No.2 with one or more nucleotides and/or deletion and/or addition and with identical function to SEQ ID No.2.

2. An application of the specific primer pair according to claim 1 in screening an apple plant with fruit storability.

3. A method for identifying storability of apple fruit, comprising the following steps:
with genomic DNA of test apple as template, conducting PCR amplification by means of the specific primer pair according to claim 1, to obtain a PCR amplified product(s): if there is one type of 655 bp PCR amplified product, a genotype of a test apple plant is homozygous for ACS1-2/-2; if there are two types of PCR amplified products of 517 bp and 655 bp, respectively, the genotype of the test apple plant is heterozygous for ACS1-1/-2; if there is one type of 517 bp PCR amplified product, the genotype of the test apple plant is homozygous for ACS1-1/-1; and
the apple plant homozygous for ACS1-2/-2 has greater fruit storability than that heterozygous for ACS1-1/-2, followed by that homozygous for ACS1-1/-1.

4. An application of the specific primer pair according to claim 1 in identifying storability of apple fruit.

5. An application of the specific primer pair according to claim 1 in apple breeding.

6. A DNA molecule, presenting a target sequence of the specific primer pair according to claim 1 in apple genome.

7. An application of the DNA molecule according to claim 6 in screening an apple plant with fruit storability.

8. A method for identifying storability of apple fruit, comprising the following steps: detecting whether there are DNA fragments depicted in SEQ ID No.3 and SEQ ID No.4 in a genomic DNA of a test apple and then determining as follows: if there is a DNA fragment depicted in SEQ ID No.3, but not in SEQ ID No.4, in the genomic DNA of the test apple, then a genotype of a test apple plant is homozygous for ACS1-2/-2; if there are DNA fragments depicted in both SEQ ID No.3 and SEQ ID No.4 in the genomic DNA of the test apple, then the genotype of the test apple plant is heterozygous for ACS1-1/-2; if there is a DNA fragment depicted in SEQ ID No.4, but not in SEQ ID No.3, in the genomic DNA of the test apple, then the genotype of the test apple plant is homozygous for AC S1-1/-1; and
the apple plant homozygous for ACS1-2/-2 has greater fruit storability than that heterozygous for ACS1-1/-2, followed by that homozygous for ACS1-1/-1.

9. An application of the DNA molecule according to claim 6 in identifying storability of apple fruit.

10. An application of the DNA molecule according to claim 6 in apple breeding.


SEQUENCE LISTING (4 sequences)

SEQ ID NO: 1 - DNA, 25 bp, Malus domestica
agagagatgccatttttgttcgtac

SEQ ID NO: 2 - DNA, 30 bp, Malus domestica
cctacaaacttgcgtggggattataagtgt

SEQ ID NO: 3 - DNA, 655 bp, Malus domestica
agagagatgccatttttgttcgtaccggattttcgaggttgactcaaatcaaaacattgtttggtaattggagtaatgaactgagcagacataaaaacctgtgcgaacttaaaaggttaaaaaaaaggttaaaaaaaaaaactttaacgaaaaaatatcagtattgttcattttaacgaaaaatcacacttttacattaaaaagtcaaccctgttactatttattttaccctttattttgtttaaaactcaaaatttttaagtatttttcattaattttctttaaaaaaaatagaaagtgagaaaaatgcccgacaaaattagttgtggctactagagtcaagaagcatatggaccagggtgggtcgctcttggcattttctatgatacttgttgtcggtaaggttttgtaaacaaaactagacccgagtattaattcttgtttcactagtacttaacagcatatttaccacatcaattccatcacgggcatcaacagctcgtggtagaaactattcaattaccacggttacatgtagatcgtggtagataacattaattcaccacggtcaaattctggcccgttgttaaaagtaaataattcaccacgattaatgcttctatgtacacttataatccccacgcaagtttgtagg

SEQ ID NO: 4 - DNA, 517 bp, Malus domestica
agagagatgccatttttgttcgtaccggattttcgaggttgactcaaatcaaaacattgtttggtaattggagtaatgaactgagcagacataaaaacctgtgcgaacttaaaggttaaaaaaaaggttaaaaaaaaaaaacttaaacgaaaaaatctcagtattgtttattttaacaaaaattcacacttttacattaaaaagtcaatcctgttattatttattttaccctttattttatttaaaactcaaagtttttaagtatttttcattaattttccttaaaaaaaatagaaagtgagaaaaatgcccgacaaaattagttgtggctactagagtcaagaagcatatggaccagggtgggtcgctcttggcattttctatgatacttgttgtcggtaaggttttgtaaacaaaactagacccgagtattaattcttgtttctttgtttttttttcaattacaagccgattaatgcttctatgtacacttataatccccacgcaagtttgtagg